(19) United States
(12) Patent Application Publication
(10) Pub. No.: US 2017/0281517 A1
(43) Pub. Date: Oct. 5, 2017

(54) SWEAT-INHIBITING AND/OR DEODORIZING COSMETIC AGENTS WITH LOW EMULSIFIER CONTENT

(71) Applicant: Henkel AG & Co. KGaA, Duesseldorf (DE)

(72) Inventors: Thomas Doering, Dormagen (DE); Stefanie Schmitz, Moenchengladbach (DE)

(73) Assignee: Henkel AG & Co. KGaA, Duesseldorf (DE)

(21) Appl. No.: 15/466,549

(22) Filed: Mar. 22, 2017

(30) Foreign Application Priority Data
Mar. 31, 2016 (DE) 102016205332.9

(51) Int. Cl. A61K 8/87; A61K 8/33; A61Q 15/00; A61K 8/84; A61K 8/06; A61K 8/49; A61K 8/26; A61K 8/28; A61K 8/44; A61K 8/34; A61K 8/365; A61K 8/36; A61K 8/86; A61K 8/37

(52) U.S. Cl. CPC A61K 8/87; A61K 8/86; A61K 8/33; A61Q 15/00; A61K 8/84; A61K 8/062; A61K 8/375; A61K 8/26; A61K 8/28; A61K 8/44; A61K 8/34; A61K 8/365; A61K 8/36; A61K 8/4926; A61K 8/345; A61K 2800/5422; A61K 2800/21; A61K 2800/262

(57) ABSTRACT

The present invention relates to cosmetic agents having a low emulsifier content which include a combination of a specific non-ionic polyurethane polymer and at least two emulsifiers having different HLB values. The aforementioned cosmetic agents, in spite of the low emulsifier content, have a high storage stability and also good applicability and dosing accuracy. In addition, these agents are particularly skin-friendly. The present invention also relates to a method for preventing and/or reducing perspiration of the body with use of a cosmetic agent according to the invention, and also to the use of the cosmetic agent according to the invention for increasing the skin compatibility and/or for reducing and/or preventing textile discoloration and/or textile stains.

### FIELD OF THE INVENTION

The present invention relates to sweat-inhibiting and/or deodorizing cosmetic agents which include at least one non-ionic polyurethane polymer and also have an emulsifier content of at most 4.9 wt. % in relation to the total weight of the cosmetic agent. The aforementioned agents have excellent skin-caring effects and also a reduced residue formation, in particular on textiles. In addition, these agents, in spite of the low emulsifier content, have excellent storage stability.

The present invention also relates to the use of a cosmetic agent according to the invention for increasing skin compatibility and/or for reducing and/or preventing textile discoloration and/or textile stains.

Lastly, the present invention relates to a non-therapeutic cosmetic agent for reducing the perspiration of the body and/or for reducing the body odor triggered by perspiration, wherein a cosmetic agent according to the invention is applied to the human skin and remains at the site of application for at least 1 hour.

### BACKGROUND OF THE INVENTION

The washing, cleaning and caring of one's own body is a basic human requirement, and modern industry is continuously attempting to satisfy this human requirement in many ways. What is particularly important for daily hygiene is the lasting elimination or at least reduction of body odor and underarm wetness.

Body odor is produced by the bacterial decomposition of the ingredients of sweat, which is initially odorless. The decomposition products, which contribute significantly to body odor, in particular to armpit body odor, can be divided into three classes: the first class is formed by short-chain C₄-C₁₀ fatty acids, which can be linear, branched, saturated and unsaturated (for example isovaleric acid, 3M2H), the second class is formed by short-chain linear or branched sulfanyl alcohols, and the third class consists of various steroid hormones and metabolic products thereof (for example 5-α-androstenol and 5-α-androstenone).

Body odor can therefore be combated by the avoidance of the bacterial breakdown of sweat. In order to avoid the bacterial breakdown of sweat, antimicrobial substances which reduce the number of sweat-decomposing bacteria on the skin by killing them off and/or which inhibit the growth of these bacteria are used in the prior art. Active substances which reduce and/or prevent the development of decomposition products by blocking bacterial enzymes are also known. In addition, it is known to absorb the volatile decomposition products by physical and/or chemical interaction and thus avoid unpleasant body odor.

Body odor can also be combated by avoidance of perspiration of the body. Cosmetic antiperspirants of the prior art include at least one sweat-inhibiting salt. In order to achieve a high sweat reduction, aluminum-zirconium halides are preferably used in the prior art. The sweat-inhibiting effect of these salts can be further increased for example by thermal treatment and addition of ligands or phosphates.

In the prior art numerous specific deodorizing or sweat-inhibiting body care agents are known which have been developed for use in areas of the body having a high density of sweat glands, in particular in the armpit area. These agents are provided in a very wide range of administration forms, for example as a powder, in stick form, as an aerosol spray, pump spray, liquid and gel-like roll-on application, cream, gel and saturated flexible substrate (deodorant wipes).

Such prior art agents, in particular based on emulsions, can lead to poorer skin compatibility on account of the high emulsifier content necessary for the stabilization of the emulsion. Furthermore, with use of such prior art agents, textile stains can form, which are considered undesirable by the consumer.

### BRIEF SUMMARY OF THE INVENTION

There is a need for cosmetic agents which have good skin compatibility and also reduced residue formation, in particular on textiles. In addition, these agents should also have an excellent sweat-inhibiting and/or deodorizing effect and also a high storage stability.

The object of the present invention was therefore to provide a cosmetic agent which avoids or at least mitigates the disadvantages of the prior art and which has high skin compatibility and storage stability and also reduced residue formation, in particular on textiles. This cosmetic agent should also have an excellent sweat-inhibiting and/or deodorizing effect. In addition, these agents should have good cosmetic properties. Furthermore, other desirable features and characteristics of the present invention will become apparent from the subsequent detailed description of the invention and the appended claims, taken in conjunction with this background of the invention.

The subject of the present invention is thus a cosmetic agent including, in a cosmetically acceptable carrier and in relation to its total weight,

a) at least one non-ionic polyurethane polymer, comprising at least one polyurethane compound of formula (I) and at least one non-ionic polyether compound of formula (II) and at least one ether compound of formula (III)

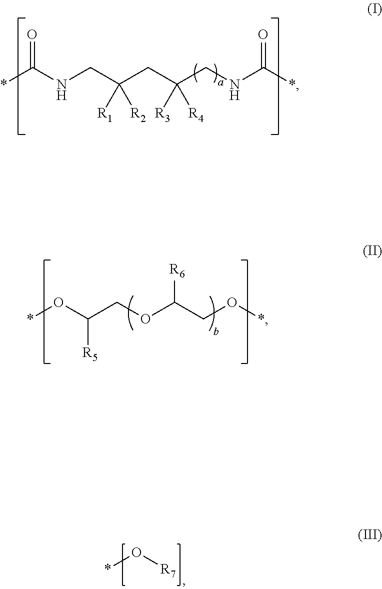

in which
R₁ to R₆, each independently of one another, stand for hydrogen or a linear or branched C₁-C₄ alkyl group,
R₇ stands for a linear or branched C₁₀-C₂₈ alkyl group,

a stands for integers from 1 to 5, and

b stands for integers from 50 to 200,

b) at least one emulsifier having an HLB value from 1 to 6,
c) at least one emulsifier having an HLB value from 10 to 17, and
d) at least one sweat-inhibiting and/or deodorizing active substance,
wherein the total amount of emulsifiers in the cosmetic agent is from 0.01 to 4.9 wt. %.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that, by use of a non-ionic polyurethane polymer and also a combination of at least two emulsifiers having different HLB values, the total amount of emulsifiers can be reduced to less than 5 wt. %, in relation to the total weight of the cosmetic agent, without negatively influencing the storage stability of these agents. Due to the low emulsifier concentration, an improved skin compatibility is achieved. In addition, the agents according to the invention lead to a reduced residue formation, in particular on textiles. In addition, these agents have excellent sweat-inhibiting and/or deodorizing and also cosmetic properties.

Without wishing to be limited to this theory, the non-ionic polyurethane polymer leads to the formation of a structured network, whereby a thickening and stabilization of the cosmetic agents according to the invention is achieved. On account of this network, the amount of emulsifiers can be significantly reduced without negatively influencing the storage stability of the cosmetic agents. Due to the use of emulsifiers having different HLB values, there is an effective emulsification of the ingredients of the cosmetic agents in spite of the low emulsifier amount, thus resulting in a high storage stability. On account of the low total amount of skin-irritating emulsifiers, the cosmetic agents according to the invention are particularly skin-friendly. In addition, these agents do not lead to textile staining, which is considered undesirable by the consumer. In addition, the agents are present in the form of a clear composition and are therefore visually particularly appealing.

The term “non-ionic polyurethane polymers” is to be understood within the scope of the present invention to mean polymers in which the monomer units are linked by urethane groupings of the general formula —NH—CO—O— and which, independently of the pH value, do not include any ionic, in particular anionic and cationic, or ionizable groups, such as amine groups. Polyurethane polymers used in accordance with the invention can be produced for example by reacting polyisocyanates (compound having at least two isocyanate groups) with polyols (compound having at least two free OH groups), diols and amines. However, it is preferred within the scope of the present invention if initially an NCO-group-terminated polyurethane prepolymer is produced by reacting an excess of a diisocyanate, for example a diisocyanate corresponding to formula (I), with a non-ionic polyetherpolyol corresponding to formula (II) and optionally a polyamine corresponding to formula (IV). This polyurethane prepolymer is then reacted with an ether compound of formula (III), wherein the free OH group of the ether compound reacts with the at least one free NCO group of the polyurethane prepolymer, thus forming a covalent bond. The ether compounds of formula (III) are therefore preferably arranged terminally in the non-ionic polyurethane polymer.

The term polyurethane compounds is to be understood to mean compounds which comprise at least two urethane groups —NH—C(O)—. Furthermore, non-ionic polyether compounds are understood to mean compounds which comprise at least two ether groups and do not include any charged groups irrespective of pH value. By contrast, ether compounds in accordance with the invention are compounds which comprise merely one ether group.

In accordance with the above formulas and all subsequent formulas, a chemical bond characterized by the symbol “*” stands for a free valence of the corresponding structure fragment. Here, a “free valence” is understood to mean the number of atomic bonds emanating from the corresponding structure fragment in the position characterized by the symbol “*”. Within the scope of the present invention, an atomic bond preferably emanates from each of the positions of the structure fragments characterized by the symbol “*” to further structure fragments.

The term “emulsifiers” is to be understood in accordance with the invention to mean compounds which can reduce the interfacial tension between the different phases of the cosmetic agents and which in this way lead to a stabilization of the cosmetic agents. Emulsifiers of this type have an amphiphilic molecular structure, i.e. they have both polar and non-polar groups. The polar groups are groups which have a hydrophilic nature. Non-polar groups are understood to be hydrophilic or lipophilic groups. These emulsifiers can therefore interact both with hydrophilic and lipophilic phases. This results in an orientation of the emulsifiers at the interface between hydrophilic and hydrophobic phase, thus resulting in a stabilization.

Within the scope of the present invention the term “HLB value” is understood to mean a measure for the water solubility or oil solubility of predominantly non-ionic surfactants, said measure having been introduced by Griffin in 1950. The HLB value can be determined experimentally for example by the phenol titration method by adding 5% phenol solution to the emulsifier solution until the mixture becomes cloudy. The HLB value can also be determined by (gas) chromatography, by determining the permittivity, or by colorimetry. The HLB value of an emulsifier mixture can be calculated additively from the values of the constituents of said mixture. The scale of the HLB values generally ranges from 1 to 20. Substances having a low HLB value of less than 8 are generally good water-in-oil emulsifiers, whereas compounds that are more hydrophilic having an HLB value of 8 and more act as oil-in-water emulsifiers.

The term “sweat-inhibiting active substance” is understood in accordance with the invention to mean active substances which lead to an elimination or reduction of the perspiration of the sweat glands of the body.

In addition, the term “deodorizing active substance” is understood in accordance with the invention to mean active substances which lead to a reduction or avoidance of the bacterial breakdown of sweat and/or which absorb or mask the malodorous volatile decomposition products. However, this term does not include ethanol used as a constituent of the carrier.

Lastly, the total amount of emulsifiers is understood to mean the total amount of all emulsifiers included in the cosmetic agent, in particular also those which do not have HLB values of 1 to 6 or 10 to 17. In particular, the total amount of emulsifiers is understood to mean the total amount of all emulsifiers having HLB values of 1 to 18 included in the agent.

The specification wt. % in the present case, unless otherwise specified, relates to the total weight of the cosmetic agents according to the invention, wherein the sum of all ingredients of the agents according to the invention gives 100 wt. %.

The cosmetic agents according to the invention include the non-ionic polyurethane polymer a), the at least one emulsifier b) having an HLB value of 1 to 6, the at least one emulsifier c) having an HLB value of 10 to 17, the at least one sweat-inhibiting and/or deodorizing active substance d), and optionally further ingredients in a cosmetically acceptable carrier.

Preferred cosmetically acceptable carriers are aqueous, alcoholic or aqueous-alcoholic media comprising preferably at least 10 wt. % water, in relation to the total weight of the cosmetic agent. The cosmetically acceptable carrier particularly preferably includes water, in particular in an amount such that the cosmetic agent includes at least 10 wt. %, preferably at least 20 wt. %, in particular at least 40 wt. % water, calculated on the total weight of the cosmetic agent. Very particularly preferred cosmetic agents have, in relation to their total weight, a water content of 50 to 95 wt. %, preferably from 60 to 90 wt. %, in particular from 65 to 85 wt. %.

In particular, the lower alcohols having 1 to 4 carbon atoms usually used for cosmetic purposes, for example ethanol and isopropanol, can be included as alcohols. Examples of water-soluble solvents as co-solvents are glycerol and/or ethylene glycol and/or 1,2-propylene glycol, which can be used in an amount of 0 to 5.0 wt. %, in relation to the total weight of the cosmetic agent.

As first essential constituent a), the cosmetic agent according to the invention includes at least one non-ionic polyurethane polymer, comprising at least one polyurethane compound of formula (I) and at least one non-ionic polyether compound of formula (II) and at least one ether compound of formula (III).

In accordance with the invention, the non-ionic polyurethane polymer advantageously includes specific polyurethane compounds of formula (I). It is therefore preferred within the scope of the present invention when the at least one non-ionic polyurethane polymer comprises, as polyurethane compound of formula (I), at least one polyurethane compound of formula (Ia) or of formula (Ib) and/or of formula (Ic)

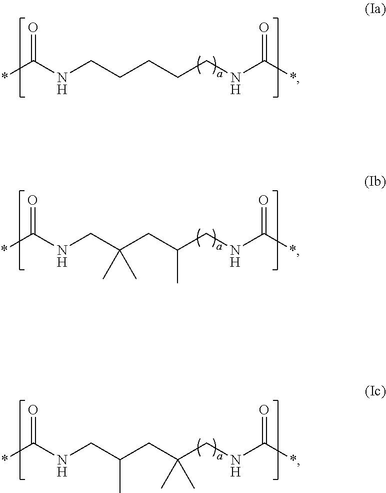

in which
a in each case stands for the integer 2.

Non-ionic polyurethane polymers which include, as polyurethane compound of formula (I), the aforementioned polyurethane compounds of formulas (Ia) or (Ib) and/or (Ic), lead to a particularly good stabilization of the cosmetic agents according to the invention. These polyurethane compounds are obtained by the use of diisocyanates, such as hexamethylene diisocyanate (HDI, formula (Ia)) or 2,2,4- and/or 2,4-4-trimethylhexamethylene diisocyanate (TMHDI, formulas (Ib) and (Ic)), during the production of the non-ionic polyurethane polymer.

It is preferred in accordance with the invention when the non-ionic polyurethane polymer, in addition to the at least one polyurethane compound of formula (I), in particular of formulas (Ia) or (Ib) and/or (Ic), additionally includes a specific non-ionic polyether compound of formula (IIa). Preferred sweat-inhibiting cosmetic agents are therefore characterized in that the at least one non-ionic polyurethane L comprises, as non-ionic polyether compound of formula (II), at least one non-ionic polyether compound of formula (IIa)

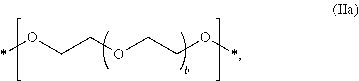

in which
b stands for integers of 60 to 150, in particular from 80 to 110.

The presence of the non-ionic polyether compounds of formula (IIa) in the form of polyethylene glycols leads, in combination with the polyurethane compounds of formula (I), in particular of formulas (Ia) or (Ib) and/or (Ic), to an improved stabilization of the cosmetic agents according to the invention and therefore to an increased storage stability.

Furthermore, it can be preferred in accordance with the invention when the non-ionic polyurethane polymer includes a specific ether compound of formula (III). Preferred sweat-inhibiting cosmetic agents are therefore characterized in that the at least one non-ionic polyurethane polymer comprises, as ether compound of formula (III), at least one ether compound of formula (IIIa) or of formula (IIIb)

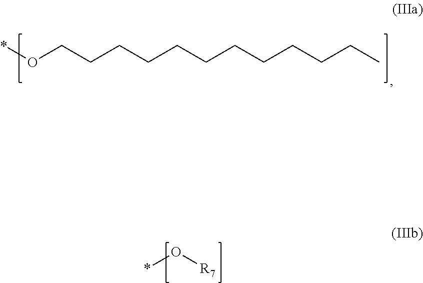

in which
R₇ stands for a branched C₁₆-C₂₀ alkyl group.

The use of non-ionic polyurethane polymers which include the aforementioned ether compounds of formulas (IIIa) or (IIIb) in combination with the polyurethane compound of formula (I), in particular of formulas (Ia) or (Ib) and/or (Ic), and also the non-ionic polyether compound of formula (II), in particular of formula (IIa), lead to a particularly effective increase of the storage stability of the cosmetic agents according to the invention, such that it is possible to dispense with the use of high emulsifier concentrations. The cosmetic agents according to the invention are therefore particularly skin-friendly.

It has proven to be advantageous within the scope of the present invention when the non-ionic polyurethane polymer, in addition to the at least one polyurethane compound of formula (I), in particular (Ia) or (Ib) and/or (Ic), the at least one non-ionic polyether compound of formula (II), in particular (IIa), and also the ether compound of formula (III), in particular (IIIa) or (IIIb), additionally includes at least one polyamine compound of formula (IV). Polyamine compounds within the scope of the present invention are to be understood to mean compounds which include at least two NR₂ groupings, wherein R stands for hydrogen or a C₁-C₄ alkyl group. Preferred cosmetic agents of the present invention are therefore characterized in that the at least one non-ionic polyurethane polymer additionally comprises at least one polyamine compound of formula (IV)

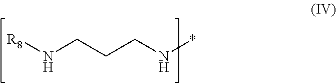

in which
R₈ stands for a linear or branched C₈-C₂₀ alkyl group.

The group R₈ is preferably a mixture of alkyl groups having different chain lengths, wherein the following distribution is particularly preferred: 6% C₈ alkyl groups, 7% C₁₀ alkyl groups, 50% C₁₂ alkyl groups, 20% C₁₄ alkyl groups, 10% C₁₆ alkyl groups, 7% C₁₈ alkyl groups.

Within the scope of the present invention it is particularly preferred when the cosmetic agents according to the invention includes specific non-ionic polyurethane polymers. Particularly preferred sweat-inhibiting cosmetic agents according to the invention are therefore characterized in that the at least one non-ionic polyurethane polymer comprises at least one polyurethane compound of formula (Ia), at least one non-ionic polyether compound of formula (IIa), at least one ether compound of formula (IIIa), and at least one polyamine compound of formula (IV),

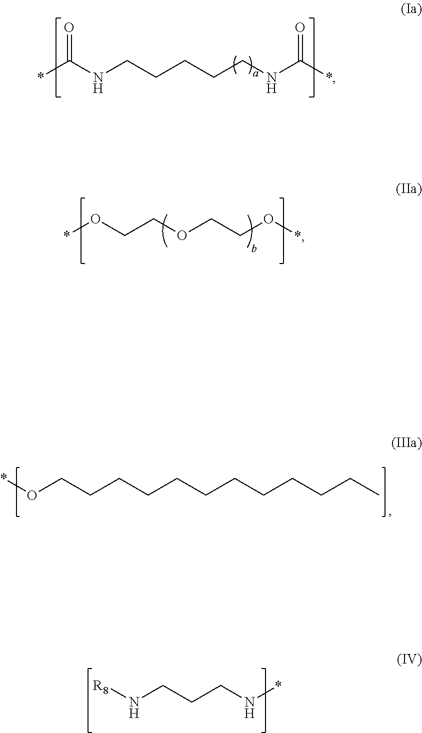

in which
a stands for the integer 2,
b stands for integers of 80 to 110, and
R₈ stands for a linear or branched C₈-C₂₀ alkyl group.

The use of non-ionic polyurethane polymers of this type in the cosmetic agents according to the invention allows the use of a particularly low total emulsifier concentration, without negatively influencing the storage stability or the cosmetic properties of the cosmetic agents. On account of the low total emulsifier concentration, these agents have excellent skin compatibility. In addition, the use of non-ionic polyurethane polymers of this type does not result in a staining on textiles, this being considered undesirable by the consumer. In addition, the use of these polyurethane polymers leads to a thickening of the cosmetic agents, such that good applicability and dosing accuracy are attained already without the addition of further thickening agents.

In particular, the use of specific non-ionic polyurethane polymers including a polyurethane compound of formula (Ia), a non-ionic polyether compound of formula (IIa), an ether compound of formula (IIIa), and a polyamine compound of formula (IV) leads to a particularly good stabilization of the ingredients and thus allows the use of less than 5 wt. % emulsifiers, such that excellent skin-caring properties of the cosmetic agents can be achieved. It is therefore particularly preferred in accordance with the invention when the at least one non-ionic polyurethane polymer is a copolymer modified terminally with lauryl alcohol and consisting of cocoaminopropylamine, polyethylene glycol having an average of 100 ethylene oxide units and hexamethylene diisocyanate (HDI). Non-ionic polyurethane polymers of this type have hydrophobic end groups in the form of lauryl groups and also a hydrophilic middle part by use of polyethylene glycol. Polyurethanes of this type are therefore capable of effectively stabilizing and cross-linking micelles formed in the cosmetic agents. On account of this stabilization, the total emulsifier concentration necessary for sufficient stability of the cosmetic agents can be significantly reduced. In addition, the cross-linking leads already to a thickening of the cosmetic agents according to the invention sufficient for good applicability and dosing accuracy, such that the use of additional thickening agents is no longer necessary.

Besides the above-described non-ionic polyurethane polymers including polyurethane compounds of formula (Ia), non-ionic polyether compounds of formula (IIa), ether compounds of formula (IIIa), and optionally polyamine compounds of formula (IV), non-ionic polyurethane compounds which include other types of polyurethane and ether compounds are also preferred. It is therefore preferred in accordance with the invention when the at least one non-ionic polyurethane polymer comprises at least one polyurethane compound of formula (Ib) and/or of formula (Ic), at least one non-ionic polyether compound of formula (IIa), and at least one ether compound of formula (IIIb)

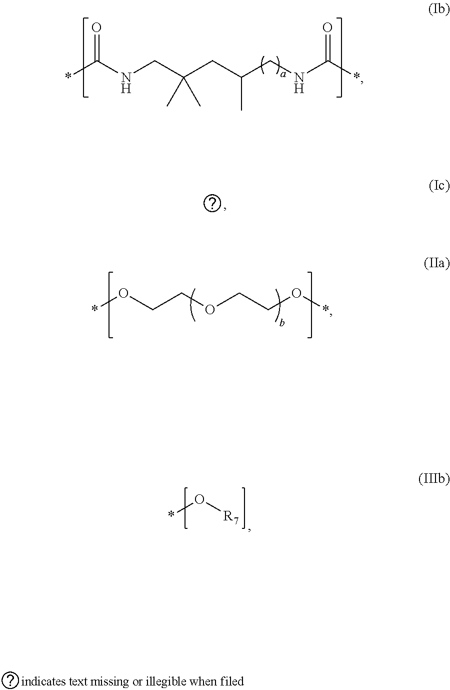

in which
a in each case stands for the integer 2,
b stands for integers of 80 to 110, and
R₇ stands for a branched C₁₆-C₂₀ alkyl group.

The use of non-ionic polyurethane polymers of this type in the cosmetic agents according to the invention allows a particularly low total emulsifier concentration without negatively influencing the storage stability or the cosmetic properties of the cosmetic agents. These agents have excellent skin compatibility on account of the low total emulsifier concentration. In addition, the use of non-ionic polyurethane polymers of this type does not result in any staining on textiles, this being considered undesirable by the consumer. In addition, the use of these polyurethane polymers leads to a thickening of the cosmetic agents, such that good applicability and dosing accuracy are attained without the addition of further thickening agents.

In particular, the use of specific non-ionic polyurethane polymers including a polyurethane compound of formula (Ib) and/or (Ic), a non-ionic polyether compound of formula (IIa), and an ether compound of formula (IIIb) lead to a particularly good stabilization of the ingredients and thus allow the use of less than 5 wt. % of emulsifiers, such that excellent skin-caring properties of the cosmetic agents can be achieved. It is therefore particularly preferred in accordance with the invention when the at least one non-ionic polyurethane polymer is a copolymer modified terminally with a branched C₁₆-C₂₀ alcohol and consisting of trimethylhexane diisocyanate (TMHDI) and polyethylene glycol having an average of 90 ethylene oxide units. Non-ionic polyurethane polymers of this type have hydrophobic end groups in the form of branched C₁₆-C₂₀ alkyl groups and also a hydrophilic middle part by use of polyethylene glycol. Polyurethanes of this type are therefore also capable of effectively stabilizing and cross-linking micelles formed in the cosmetic agents. On account of this stabilization, the total emulsifier concentration necessary for sufficient stability of the cosmetic agents can be significantly reduced. In addition, the cross-linking leads already to a thickening of the cosmetic agents according to the invention sufficient for good applicability and dosing accuracy, such that the use of additional thickening agents is no longer necessary.

The at least one non-ionic polyurethane, in particular the above-described preferred polyurethane polymers, are preferably used in specific amount ranges. It is therefore advantageous in accordance with the invention when the at least one non-ionic polyurethane polymer is included in a total amount of 0.2 to 5.0 wt. %, preferably from 0.5 to 4.8 wt. %, preferably from 0.7 to 4.5 wt. %, in particular from 1.0 to 4.0 wt. %, in each case in relation to the total weight of the cosmetic agent. The use of the aforementioned total amounts of the non-ionic polyurethane polymer, in particular the above-described preferred polyurethane polymers, leads to an excellent stabilization of the cosmetic agents according to the invention without the occurrence of incompatibilities with further ingredients or excessive residue formation on textiles.

As second essential constituent b), the cosmetic agent according to the invention includes at least one emulsifier having an HLB value of 1 to 6.

Within the scope of the present invention, it is preferred when specific emulsifiers having an HLB value of 1 to 6 are used. Preferred cosmetic agents according to the invention are therefore characterized in that the at least one emulsifier having an HLB value of 1 to 6 is selected from the group of mono- and diesters of stearic acid with ethylene oxide, mono- and/or di- and/or triesters of sorbitan with oleic acid, stearic acid or isostearic acid, mono- and diesters of glycerol with stearic acid and lauric acid, esters of isostearic acid with propylene oxide, lecithins, ethoxylated C₁₂-C₂₀ alcohols with on average 2 mol alcohol per mol ethylene oxide, block copolymers of ethylene oxide with polyhydroxystearate, diesters of lauric acid with 2 mol ethylene oxide per mol acid, esters of methyl glucose with stearic acid and mixtures thereof, in particular esters of isostearic acid with propylene oxide.

The mono- and diesters of stearic acid with ethylene oxide used within the scope of the present invention are, for example, the compounds known under the INCI names Glycol Distearate (HLB value=1, CAS-Nr.: 627-83-8) and Glycol Stearate (HLB value=2.9, CAS-Nr.: 111-60-4). Emulsifiers having an HLB value of 1 to 6 in the form of mono- and/or di- and/or triesters of sorbitan with oleic acid, stearic acid or isostearic acid that can be used within the scope of the present invention are, for example, the compounds known under the INCI names Sorbitan Trioleate (HLB value=1.8; CAS-Nr.: 26266-58-0), Sorbitan Sesquioleate (HLB value=3.7; CAS-Nr.: 8007-43-0), Sorbitan Oleate (HLB value=4.3; CAS-Nr.: 1338-43-8), Sorbitan Isostearate (HLB value=4.7; CAS-Nr.: 54392-26-6) and Sorbitan Stearate (HLB value=4.7; CAS-Nr.: 1338-41-6). Suitable mono- and diesters of glycerol with stearic acid and lauric acid are, for example, the compounds known under the INCI names Glyceryl Stearate (HLB value=3.8 to 5.8; CAS-Nr.: 11099-07-3, 86904-15-6, 85666-92-8, 31566-31-1) and Glyceryl Laurate (HLB value=5.2; CAS-Nr.: 27215-38-4). An ester of isostearic acid with propylene oxide that can be used in accordance with the invention is, for example, the compound known under the INCI name Propylene Glycol Isostearate (HLB value=2.5; CAS-Nr.: 63799-53-1). In addition, lecithins which have HLB values in the claimed range of 1 to 6 are also suitable as emulsifiers having an HLB value of 1 to 6. The compounds known under the INCI names Steareth-2 (HLB value=4.9; CAS-Nr.: 16057-43-5), Oleth-2 (HLB value=4.9; CAS-Nr.: 9004-98-2) and Ceteth-2 (HLB value=5.3; CAS-Nr.: 5274-61-3) are examples of ethoxylated C₁₂-C₂₀ alcohols with on average 2 mol alcohol per mol ethylene oxide that can be used in accordance with the invention. Also suitable as emulsifiers having an HLB value of 1 to 6 are block copolymers of ethylene oxide with polyhydroxystearate, which are commercially obtainable for example under the INCI name PEG-30 Dipolyhydroxystearate (HLB value=5.5; CAS-Nr.: 70142-34-6). In addition, diesters of lauric acid with 2 mol ethylene oxide per mol acid can be used in accordance with the invention and are known for example under the INCI name PEG-4 Dilaurate (HLB value=6; CAS-Nr.: 10108-24-4). Lastly, esters of methyl glucose with stearic acid can also be used in accordance with the invention and are known for example under the INCI name Methyl Glucose Sesquistearate (HLB value=6.6: CAS-Nr.: 68936-95-8). The use of emulsifiers having an HLB value of 1 to 6 in the form of esters of isostearic acid with propylene oxide, in particular the use of 2-hydroxypropyl-16-methylheptadecanoate, is particularly preferred within the scope of the present invention.

The at least one emulsifier b) having an HLB value of 1 to 6, preferably the esters of isostearic acid with propylene oxide, in particular 2-hydroxypropyl-16-methylheptadecanoate, are preferably used in specific amount ranges. Preferred cosmetic agents according to the invention are therefore characterized in that the at least one emulsifier having an HLB value of 1 to 6 is included in a total amount of 0.1 to 1.7 wt. %, preferably from 0.2 to 1.5 wt. %, preferably from 0.3 to 1.2 wt. %, in particular from 0.4 to 1.0 wt. %, in each case in relation to the total weight of the cosmetic agent. The aforementioned amounts relate here to the total amount of emulsifiers having an HLB value of 1 to 6. Thus, if 2 or more emulsifiers having an HLB value of 1 to 6 are used, the aforementioned amount values relate to the total amount thereof. The use of the aforementioned amounts of the at least one emulsifier b) having an HLB value of 1 to 6, preferably in the form of esters of isostearic acid with propylene oxide, in particular in the form of 2-hydroxypropyl-16-methylheptadecanoate, in combination with the at least one emulsifier having an HLB value of 10 to 17 has proven to be advantageous with regard to the stability of the cosmetic agents according to the invention at emulsifier concentrations of less than 5 wt. %. On account of the extremely low emulsifier concentration, the cosmetic agents according to the invention have high skin compatibility. In addition, the use of these emulsifier amounts does not lead to an undesirable staining on textiles.

As third essential constituent c), the cosmetic agent according to the invention includes at least one emulsifier c) having an HLB value of 10 to 17.

Within the scope of the present invention, it is preferred when specific emulsifiers having an HLB value of 10 to 17 are used. Preferred cosmetic agents according to the invention are therefore characterized in that the at least one emulsifier having an HLB value of 10 to 17 is selected from the group of mono- and/or di- and/or triglycerides of coconut oil with on average 7 mol ethylene oxide per mol glyceride, mono- and/or diglycerides of almond oil with on average 20 to 60 mol ethylene oxide per mol glyceride, hydrogenated castor oil with on average 25 mol ethylene oxide per mol hydrogenated castor oil, N-(2-hydroxyethyl)octadecanamide, mono- and/or sorbitan trioleates with on average 20 mol ethylene oxide per mol sorbitan, sorbitan monostearates and/or sorbitan monolaurates with on average 20 mol ethylene oxide per mol sorbitan, ethoxylated fatty acids from olive oil with on average 7 mol ethylene oxide per mol fatty acid, C₁₆-C₁₈ alkylglucosides, esters of oleic acid and/or lauric acid with on average 8 mol ethylene oxide per mol acid, polymers of methyl-D-glucopyranoside dioctadecanoate with glycerol, N-(2-hydroxyethyl)dodecanamide, methyl-β-D-glucoside sesquistearates with on average 20 mol ethylene oxide per mol glucose ester, ethoxylated linear or branched C₁₀-C₂₂ alcohols with on average 10 to 23 mol ethylene oxide per mol alcohol, and mixtures thereof, in particular ethoxylated linear or branched C₁₀-C₂₂ alcohols with on average 10 to 23 mol ethylene oxide per mol alcohol. The mono- and/or di- and/or triglycerides of coconut oil with on average 7 mol ethylene oxide per mol glyceride that can be used within the scope of the present invention are, for example, the compound known under the INCI name PEG-7 Glyceryl Cocoate (HLB value=10; CAS-Nr.: 68201-46-7). Emulsifiers having an HLB value of 10 to 17 in the form of mono- and/or diglycerides of almond oil with on average 20 to 60 mol ethylene oxide per mol glyceride that can be used within the scope of the invention are, for example, the compounds known under the INCI names PEG-20 Almond Glycerides (HLB value=10; CAS-Nr.: 124046-50-0) and PEG-60 Almond Glycerides (HLB value=15; CAS-Nr.: 226993-90-4). Suitable emulsifiers are also hydrogenated castor oil with on average 25 mol ethylene oxide per mol hydrogenated castor oil, which is known under the INCI name PEG-25 Hydrogenated Castor Oil (HLB value=10.8; CAS-Nr.: 61788-85-0). Sorbitan mono- and/or trioleates with on average 20 mol ethylene oxide per mol sorbitan that can be used in accordance with the invention are, for example, the compounds known under the INCI names Polysorbate 80 (HLB value=15; CAS-Nr.: 9005-65-6) and Polysorbate 85 (HLB value=11; CAS-Nr.: 9005-70-3). In addition, sorbitan monostearates and/or sorbitan monolaurates with on average 20 mol ethylene oxide per mol sorbitan are also suitable as emulsifiers having an HLB value of 10 to 17 and are known for example under the INCI names Polysorbate 60 (HLB value=14.9; CAS-Nr.: 9005-67-8) and Polysorbate 20 (HLB value=16.7; CAS-Nr.: 9005-64-5). The compounds known under the INCI names Oleth-10 (HLB value=12.4; CAS-Nr.: 9004-98-2), Ceteth-10 (HLB value=12.9; CAS-Nr.: 9004-95-9), Isosteareth-20 (HLB value=15; CAS-Nr.: 52292-17-8), Ceteareth-20 (HLB value=15.2; CAS-Nr.: 68439-49-6), Oleth-20 (HLB value=15.3; CAS-Nr.: 9004-98-2), Steareth-20 (HLB value=15.3; CAS-Nr.: 9005-00-9), Steareth-21 (HLB value=15.5; CAS-Nr.: 9005-00-9), Ceteth-20 (HLB value=15.7; CAS-Nr.: 9004-95-9), Isoceteth-20 (HLB value=15.7; CAS-Nr.: 69364-63-2 and 9004-95-9) and also Laureth-23 (HLB value=16.9; CAS-Nr. 9002-92-0) are examples of ethoxylated linear or branched C₁₀-C₂₂ alcohols with on average 10 to 23 mol ethylene oxide per mol alcohol that can be used in accordance with the invention. Ethoxylated fatty acids from olive oil with on average 7 mol ethylene oxide per mol fatty acid and also C₁₆-C₁₈ alkylglucosides, which for example are known under the INCI names PEG-7 Olivate (HLB value=11; CAS-Nr.: 226708-41-4) and Cetearyl Glucoside (HLB value=11; CAS-Nr.: 54549-27-8 (C16) 27836-65-3 (C18)), are also suitable as emulsifiers having an HLB value of 10 to 17. In addition, esters of oleic acid and/or lauric acid with on average 8 mol ethylene oxide per mol acid and also polymers of methyl-D-glucopyranoside dioctadecanoate with glycerol can be used in accordance with the invention and are known for example under the INCI names PEG-8 Oleate (HLB value=11.6; CAS-Nr.: 9004-96-0), PEG-8 Laurate (HLB value=13; CAS-Nr.: 9004-81-3) and Polyglyceryl-3 Methyglucose Distearate (HLB value=12; CAS-Nr.: 68986-95-8). Lastly, methyl-β-D-glucoside sesquistearates with on average 20 mol ethylene oxide per mol glucose ester can also be used in accordance with the invention and are known for example under the INCI name PEG-20 Methyl Glucose Sesquistearate (HLB value=15; CAS-Nr.: 68389-70-8). What is preferred within the scope of the present invention is the use of at least two emulsifiers having an HLB value of 10 to 17 in the form of ethoxylated linear or branched C₁₀-C₂₂ alcohols with on average 10 to 23 mol ethylene oxide per mol alcohol. An ethoxylated branched C₁₆ alcohol with on average 20 mol ethylene oxide per mol alcohol in combination with an ethoxylated linear C₁₈ alcohol with on average 20 mol ethylene oxide per mol alcohol are particularly preferably used.

It has proven to be advantageous within the scope of the present invention to use the at least one emulsifier c) having an HLB value of 10 to 17, in particular at least two emulsifiers in the form of ethoxylated linear or branched C₁₀-C₂₂ alcohols having on average 10 to 23 mol ethylene oxide per mol alcohol, within specific amount ranges. Preferred cosmetic agents of the present invention are therefore characterized in that the at least one emulsifier having an HLB value of 10 to 17 is included in a total amount of 1.0 to 4.3 wt. %, preferably from 1.5 to 4.2 wt. %, preferably from 1.8 to 4.1 wt. %, in particular from 2.0 to 4.0 wt. %, in each case in relation to the total weight of the cosmetic agent. The aforementioned amounts relate here to the total amount of emulsifiers having an HLB value of 10 to 17. If 2 or more emulsifiers having an HLB value of 10 to 17 are used, the aforementioned amount values thus relate to the total amount thereof. The use of the aforementioned amounts of the at least one emulsifier c) having an HLB value of 10 to 17, in particular in the form of ethoxylated linear or branched C₁₀-C₂₂ alcohols having on average 10 to 23 mol ethylene oxide per mol alcohol, in combination with the at least one emulsifier having an HLB value of 1 to 6 has proven to be advantageous in respect of the stability of the cosmetic agents according to the invention at emulsifier concentrations of less than 5 wt. %. On account of the extremely low emulsifier concentration, the cosmetic agents according to the invention have high skin compatibility. In addition, the use of these emulsifier amounts does not lead to an undesirable staining on textiles.

It has proven to be advantageous in accordance with the invention when a specific ratio by weight of the at least one emulsifier c) having an HLB value of 10 to 17 to the at least one emulsifier b) having an HLB value of 1 to 6 is included in the cosmetic agents according to the invention. It is therefore preferred within the scope of the present invention when the cosmetic agent has a ratio by weight of the at least one emulsifier having an HLB value of 10 to 17 to the at least one emulsifier having an HLB value of 1 to 6 of 10:1 to 1:5, preferably from 10:1 to 1:1, preferably from 8:1 to 3:1, in particular from 7:1 to 4:1. The use of the aforementioned ratios by weight leads to an improved storage stability, such that only a low emulsifier concentration is necessary for stable emulsification of the ingredients of the cosmetic agents. On account of the low emulsifier concentration, the cosmetic agents according to the invention have high skin compatibility and excellent cosmetic properties.

Essential constituent d), the cosmetic agent according to the invention includes at least one sweat-inhibiting and/or deodorizing active substance.

Preferred sweat-inhibiting active substances within the scope of the present invention are sweat-inhibiting aluminum salts and/or aluminum zirconium salts. Advantageous cosmetic agents according to the invention are therefore characterized in that the at least one sweat-inhibiting active substance is selected from the group of (i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble astringent organic salts of aluminum, in particular aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum chlorohydroxylactate, aluminum lipo-amino acids, aluminum lactate, aluminum chlorohydroxyallantoinate, sodium aluminum chlorohydroxylactate; (iii) water-soluble astringent inorganic aluminum-zirconium salts, especially aluminum zirconium trichlorohydrate, aluminum zirconium tetrachlorohydrate, aluminum zirconium pentachlorohydrate, aluminum zirconium octachlorohydrate; (iv) water-soluble astringent organic aluminum-zirconium salts, in particular aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine; as well as (v) mixtures thereof.

The term “sweat-inhibiting aluminum salts” is understood in accordance with the invention to rule out aluminosilicates and zeolites. Furthermore, in accordance with the invention, water-soluble aluminum salts are understood to mean salts that have a solubility of at least 3 wt. % at 20° C., i.e. dissolve at least 3 g of these sweat-inhibiting aluminum salt in 97 g of water at 20° C.

Particularly preferred inorganic aluminum salts are selected from aluminum chlorohydrate, in particular aluminum chlorohydrate having the general formula [Al₂(OH)₅Cl.1-6H₂O]ₙ, preferably [Al₂(OH)₅Cl.2-3H₂O]ₙ, which can be present in the non-activated (polymerized) or in the activated (depolymerized) form, and also aluminum chlorohydrate having the general formula [Al₂(OH)₄Cl₂.1-6H₂O]ₙ, preferably [Al₂(OH)₄Cl₂.2-3H₂O]ₙ, which can be present in the non-activated (polymerized) or in the activated (depolymerized) form.

In accordance with the invention, particularly preferred sweat-inhibiting aluminum salts are selected from what are known as “activated” aluminum salts, which are also referred to as antiperspirant active substances “with enhanced activity”. Such active substances are known in the prior art and are also commercially obtainable. Activated aluminum salts typically have an HPLC peak 4 to peak 3 area ratio of at least 0.4, preferably of at least 0.7, in particular of at least 0.9, wherein at least 70% of the aluminum is attributed to these HPLC peaks.

In this context, “activated” aluminum-zirconium salts are also known, having a high HPLC peak 5 aluminum content, in particular a peak 5 area of at least 33%, preferably at least 45%, based on the total area under peaks 2 to 5, as measured by HPLC of a 10 wt. % aqueous solution of the active substance under conditions in which the aluminum species are dissolved in at least 4 successive peaks (denoted peaks 2 to 5). Preferred aluminum-zirconium salts having a high HPLC peak 5 aluminum content are also referred to as “E⁵AZCH”. Furthermore, the aforementioned activated aluminum-zirconium salt can be stabilized additionally with a water-soluble strontium salt and/or with a water-soluble calcium salt.

It is also possible in accordance with the invention to use sweat-inhibiting aluminum salts as non-aqueous solutions or solubilizates of an activated sweat-inhibiting aluminum salt or aluminum-zirconium salt. Such aluminum or aluminum-zirconium salts are stabilized against the loss of activation of the salt by the addition of an effective amount of a polyvalent alcohol having 3 to 6 carbon atoms and 3 to 6 hydroxyl groups, preferably propylene glycol, sorbitol and pentaerythritol. Complexes of activated sweat-inhibiting aluminum or aluminum-zirconium salts with a polyvalent alcohol which include 20 to 50 wt. %, preferably 20 to 42 wt. %, of activated sweat-inhibiting aluminum or aluminum-zirconium salt and 2 to 16 wt. % of molecularly bound water, wherein the balance to 100 wt. % is at least one polyvalent alcohol having 3 to 6 carbon atoms and 3 to 6 hydroxyl groups, are also particularly preferred. Propylene glycol, propylene glycol/sorbitol mixtures and propylene glycol/pentaerythritol mixtures are preferred such alcohols.

Within the scope of the present invention it is also possible to use basic calcium-aluminum salts as sweat-inhibiting aluminum salts. These salts can be obtained by reacting calcium carbonate with aluminum chlorohydroxide or aluminum chloride and aluminum powder or by adding calcium chloride dihydrate to aluminum chlorohydroxide. However, it is also possible to use aluminum-zirconium complexes which are buffered with salts of amino acids, in particular with alkaline and alkaline earth glycinates.

Aluminum salts or aluminum-zirconium salts which are preferably stabilized by amino acids, in particular glycine, hydroxyalkanoic acids, in particular glycolic acid and lactic acid, or betaines, can also be used as preferred sweat-inhibiting activated aluminum salts and aluminum-zirconium salts in accordance with the invention.

Further preferred activated aluminum salts are those having the general formula Al₂(OH)₆₋ₐXa, in which X stands for Cl, Br, I or NO₃ and “a” stands for a number from 0.3 to 5, preferably from 0.8 to 2.5 in particular from 1 to 2, such that the molar ratio of Al:X is 0.9:1 to 2.1:1. Aluminum chlorohydrate is particularly preferred (i.e. X in the aforementioned formula stands for Cl) and especially ⅚ basic aluminum chlorohydrate with “a”=1, such that the molar ratio of aluminum to chlorine is 1.9:1 to 2.1:1.

Preferred activated aluminum-zirconium salts are those of the general formula ZrO(OH)_{2-pb}Y_{b}, in which Y stands for Cl, Br, I, NO₃ or SO₄, b stands for a rational number from 0.8 to 2, and p stands for the valence of Y, such that the Al:Zr molar ratio is from 2 to 10 and the metal:(X+Y) ratio is from 0.73 to 2.1, preferably from 0.9 to 1.5. Such activated sweat-inhibiting aluminum-zirconium salts are disclosed for example in the aforementioned document U.S. Pat. No. 6,074,632 A. A particularly preferred salt is aluminum-zirconium chlorohydrate (i.e. X and Y stand for Cl), which has an Al:Zr ratio of 2 to 10 and a molar metal:Cl ratio of 0.9 to 2.1.

In accordance with the invention particularly preferred sweat-inhibiting aluminum salts have a molar metal-to-chloride ratio of 1.9 to 2.1. The metal-to-chloride ratio of aluminum sesquichlorohydrates, which are also particularly preferred within the scope of the invention, is 1.5:1 to 1.8:1. Preferred aluminum-zirconium tetrachlorohydrates have a molar ratio of Al:Zr from 2 to 6 and of metal:chloride from 0.9 to 1.3, wherein, in particular, salts with a metal- to chloride molar ratio from 0.9 to 1.1, preferably from 0.9 to 1.0, are preferred.

The sweat-inhibiting active substance, in particular an aforementioned aluminum and/or aluminum-zirconium salt, is preferably used in specific amount ranges. Preferred cosmetic agents according to the invention are therefore characterized in that the at least one sweat-inhibiting active substance is included in a total amount of 2.0 to 40 wt. %, preferably from 3.0 to 35 wt. %, preferably from 4.0 to 32 wt. percent, more preferably from 5.0 to 28 wt. percent, even more preferably from 8.0 to 25 wt. %, in particular from 12 to 22 wt. %, in each case in relation to the total weight of the cosmetic agent. The use of the aforementioned amounts of the at least one sweat-inhibiting active substance ensures a sufficient sweat-inhibiting effect and does not lead to skin incompatibilities or negative interactions with other ingredients of the agents according to the invention. In addition, with use of these amounts, a negative influence on the cosmetic properties and also staining on textiles is avoided.

In addition or instead of the aforementioned sweat-inhibiting active substance, the cosmetic agents can include at least one deodorizing active substance.

It is advantageous within the scope of the present invention when the at least one deodorizing active substance is selected from the group of (i) silver salts; (ii) aromatic alcohols, especially 2-benzyl-heptan-1-ol, and mixtures of 2-benzylheptan-1-ol and phenoxyethanol; (iii) 1,2-alkanediols having 5 to 12 carbon atoms, in particular 3-(2-ethylhexyloxy)-1,2-propanediol; (iv) triethyl citrates; (v) active substances against exoesterases, in particular against arylsulfatase, lipase, beta-glucuronidase and β-lyase cystathione; (vi) cationic phospholipids; (vii) odor absorbers, in particular silicates, such as montmorillonite, kaolinite, illite, beidellite, nontronite, saponite, hectorite, bentonite, smectite, and talc, zeolites, zinc ricinoleate, cyclodextrins; (viii) deodorizing ion exchangers; (ix) germ inhibitors; (x) probiotically active components; and (xi) mixtures thereof.

The deodorizing active substance is preferably used within specific amount ranges. Preferred cosmetic agents according to the invention are therefore characterized in that the at least one deodorizing active substance is included in a total amount of 0.0001 to 40 wt. %, preferably from 0.2 to 20 wt. percent, preferably from 1.0 to 15 wt. %, in particular from 1.5 to 5.0 wt. %, in each case in relation to the total weight of the cosmetic agent. The use of the aforementioned amounts of the at least one deodorizing active substance ensures a sufficient deodorizing effect and does not lead to any negative interactions with other ingredients of the agents according to the invention.

It has proven to be particularly advantageous within the scope of the present invention when the cosmetic agent has only an extremely low total concentration of emulsifiers. In particular, the skin compatibility and also the reduction of textile stains can be significantly improved as a result. It is therefore particularly preferred in accordance with the invention when the total amount of emulsifiers in the cosmetic agent is from 0.05 to 4.9 wt. %, preferably from 0.1 to 4.9 wt. %, preferably from 0.3 to 4.9 wt. %, in particular from 0.5 to 4.9 wt. %, in each case in relation to the total weight of the cosmetic agent.

The cosmetic agents according to the invention can be present in the form of emulsions (O/W or W/O emulsions), aqueous, aqueous-alcoholic or aqueous-glycolic solutions, and in the form of solid compositions. It has proven to be particularly advantageous in accordance with the invention when the cosmetic agents are present in the form of emulsions. Particularly preferred cosmetic agents are therefore characterized in that they are present in the form of a transparent O/W emulsion having a mean particle diameter D₅₀ of 10 to 100 nm. The mean particle diameter of the particles or droplets present in the emulsion can be determined by means of laser diffraction, for example. The term transparent O/W emulsions is understood to mean emulsions which have an NTU value (nephelometric turbidity unit value) of 0 to 30, in particular from 0 to 20. The NTU value here is a measurement value for transparency and is a turbidity of the emulsion measured using a calibrated nephelometer. Clouded emulsions have NTU values of more than 30, whereas transparent emulsions have NTU values of 30 and below. The NTU value of the O/W emulsions according to the invention can be determined for example by means of a turbidimeter. O/W emulsions which have the aforementioned particle sizes are referred to within the scope of the present invention as micro emulsions. By using the at least one non-ionic polyurethane polymer, the oil droplets in the oil phase can be effectively cross-linked and stabilized, such that a stable O/W emulsion can be produced already with an extremely low emulsifier content. On account of the only very small amount of skin-irritating emulsifiers, the cosmetic agents according to the invention in the form of transparent O/W emulsions have excellent skin compatibility.

Cosmetic agents in the form of O/W emulsions include an oil phase including at least one compound selected from the group of (i) volatile non-silicone oils, in particular liquid paraffin oils and isoparaffin oils, such as isodecane, isoundecane, isododecane, isotridecane, isotetradecane, isopentadecane, isohexadecane and isoeicosane; (iii) non-volatile non-silicone oils, in particular the esters of linear or branched saturated or unsaturated C₂₋₃₀ fatty alcohols with linear or branched saturated or unsaturated C₂₋₃₀ fatty acids, which may be hydroxylated, the C₈-C₂₂ fatty alcohol esters of monovalent or polyvalent C₂-C₇ hydroxycarboxylic acids, the triethyl citrates, the branched saturated or unsaturated C₆₋₃₀ fatty alcohols, the mono-, di- and triglycerides of linear or branched, saturated or unsaturated, optionally hydroxylated C₈₋₃₀ fatty acids, the dicarboxylic acid esters of linear or branched C₂-C₁₀ alkanols, the addition products of ethylene oxide and/or propylene oxide with mono- or polyvalent C₃₋₂₂ alkanols, which may be optionally esterified, the symmetrical, asymmetrical or cyclic esters of carbonic acid with fatty alcohols, the esters of dimers of unsaturated C₁₂₋₂₂ fatty acids with monovalent linear, branched and cyclic C₂₋₁₈ alkanols or C₂₋₆ alkanols, the benzoic acid esters of linear or branched C₈₋₂₂ alkanols, such as benzoic acid C₁₂₋₁₅ alkyl esters and isostearyl benzoate and octyldodecyl benzoate esters, the synthetic hydrocarbons such as polyisobutene and polydecene, the alicyclic hydrocarbons; and (iii) mixtures thereof

Preferred volatile non-silicone oils in accordance with the invention are those in the form of C₁₀₋₁₃ isoparaffin mixtures with a vapor pressure of 10 to 400 Pa (0.08 to 3 mm Hg), preferably from 13 to 100 Pa (0.1 to 0.8 mm Hg), at 20° C. and an ambient pressure of 1,013 hPa.

In this context, preferred cosmetic agents according to the invention include at least one ester of the linear or branched saturated or unsaturated fatty alcohols having 2 to 30 carbon atoms with linear or branched saturated or unsaturated fatty acids having 2 to 13 carbon atoms, which can be hydroxylated, in a total amount of 0.1 to 5.0 wt. %, preferably from 0.3 to 4.0 wt. %, preferably from 0.5 to 3.5 wt. %, in particular from 0.6 to 2.5 wt. %, in relation to the total weight of the cosmetic agent.

O/W emulsions according to the invention can be produced by methods known in the prior art.

In the tables on pages 22 and 23 of the priority document, particularly preferred agents K 1 to K 48 of the cosmetic agents according to the invention are specified, which are preferably present in the form of a transparent O/W emulsion having a mean particle diameter D₅₀ of 10 to 100 nm (all values are in wt. %, unless specified otherwise; nio. PU polymer=non-ionic polyurethane polymer a); total emulsifier content is in each case 0.01 to 4.9 wt. %, in relation to the total weight of the corresponding cosmetic agent).

In a particularly preferred embodiment AF 1 the aforementioned cosmetic agents K 1 to K 48 have a total emulsifier content of 0.5 to 4.9 wt. %, in each case in relation to the total weight of the corresponding cosmetic agent.

In a further, particularly preferred embodiment AF 2, the aforementioned cosmetic agents K 1 to K 48 have a ratio by weight of the at least one emulsifier having an HLB value of 10 to 17 to the at least one emulsifier having an HLB value of 1 to 6 of 7:1 to 4:1 and also a total emulsifier content of 0.5 to 4.9 wt. %, in each case in relation to the total weight of the corresponding cosmetic agent.

In addition, a particularly preferred embodiment AF 3 within the scope of the present invention is characterized in that the cosmetic agents K 1 to K 48 and the cosmetic agents of the aforementioned particularly preferred embodiments AF 1 and AF 2 include, as sweat-inhibiting active substance, a sweat-inhibiting aluminum and/or aluminum-zirconium salt, in particular aluminum chlorohydrate, aluminum sesquichlorohydrate, aluminum dichlorohydrate, aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine, and mixtures thereof

Furthermore, a particularly preferred embodiment AF 4 within the scope of the present invention is characterized in that the cosmetic agents K 1 to K 48 and also the cosmetic agents of the aforementioned particularly preferred embodiments AF 1 and AF 2 include, as deodorizing active substance, 3-(2-ethylhexyloxy)-1,2-propanediol, 1,2-hexanediol and/or 1,2-octanediol, octenidine, 2-butyloctanoic acid, triethyl citrate, phenoxyethanol, and mixtures thereof

The aforementioned cosmetic agents K 1 to K 48 and also the aforementioned particularly preferred embodiments AF 1 to AF 3 have an extremely low emulsifier content due to the use of specific non-ionic polyurethane polymers and are therefore particularly skin-compatible. In spite of the low emulsifier content, these agents and embodiments have a high storage stability, which can be increased further by the use of a specific emulsifier combination. In addition, the agents and embodiments, due to the use of the thickening non-ionic polyurethane polymer, are present already with a viscosity that is optimal for applicability and dosing accuracy, such that it is possible to dispense with the use of additional thickening agents. Lastly, transparent and thus visually appealing cosmetic agents can be produced with the aforementioned combination.

A second subject of the present invention is the use of a cosmetic agent according to the invention for increasing the skin compatibility and/or for reducing and/or preventing textile discoloration and/or textile stains. Due to the use of the non-ionic polyurethane polymer in combination with emulsifiers having different HLB values, the total emulsifier concentration of the cosmetic agents can be significantly reduced. The cosmetic agents according to the invention have improved skin compatibility on account of the lower amount of skin-irritating emulsifiers. In addition, by means of the aforementioned combination, visually appealing, clear compositions can be formulated, which already have a viscosity necessary for good applicability and dosing accuracy, such that the addition of further thickening agents is unnecessary.

That which has been said already in respect of the cosmetic agents according to the invention applies, mutatis mutandis, in respect of further embodiments of the use according to the invention, in particular in respect of the used cosmetic agent according to the invention.

Lastly, a third subject of the present invention is a non-therapeutic cosmetic method for reducing the perspiration of the body and/or for reducing the body odor triggered by perspiration, wherein a cosmetic agent according to the invention is applied to the human skin and remains at the site of application for at least 1 hour.

That which has been said already in respect of the cosmetic agents according to the invention and the use according to the invention applies, mutatis mutandis, in respect of further embodiments of the method according to the invention, in particular in respect of the cosmetic agent used there.

The following examples explain the present invention, but do not limit it thereto:

### Examples

The emulsifier used in the following examples having an HLB value of 1 to 6 is preferably 2-hydroxypropyl-16-methylheptadecanoate. The used emulsifier having an HLB value of 10 to 17 is preferably also a mixture of an ethoxylated branched C₁₆ alcohol with on average 20 mol ethylene oxide per mol alcohol and an ethoxylated linear C₁₈ alcohol with on average 20 mol ethylene oxide per mol alcohol.

The sweat-inhibiting active substance is preferably a sweat-inhibiting aluminum and/or aluminum-zirconium salt, in particular aluminum chlorohydrate, aluminum sesquichlorohydrate, aluminum dichlorohydrate, aluminum zirconium propylene glycol complexes, aluminum zirconium trichlorohydrex glycine, aluminum zirconium tetrachlorohydrex glycine, aluminum zirconium pentachlorohydrex glycine, aluminum zirconium octachlorohydrex glycine, and mixtures thereof. The following are preferably used as deodorizing active substance: 3-(2-ethylhexyloxy)-1,2-propanediol, 1,2-hexanediol and/or 1,2-octanediol, octenidine, 2-butyloctanoic acid, triethyl citrate, phenoxyethanol, and mixtures thereof

The non-ionic polyurethane polymer A preferably comprises at least one polyurethane compound of formula (Ia), at least one non-ionic polyether compound of formula (IIa), at least one ether compound of formula (IIIa), and at least one polyamine compound of formula (IV) having the above-described groups. The non-ionic polyurethane polymer B preferably comprises at least one polyurethane compound of formula (Ib) and/or (Ic), at least one non-ionic polyether compound of formula (IIa), and at least one ether compound of formula (IIIb) having the above-described groups.

The following amount values are specified in wt. %, in relation to the total weight of the corresponding cosmetic agents according to the invention.

| | Raw material | 1 | 2 | 3 | 4 | 5 | 6 |
|---|---|---|---|---|---|---|---|
| A | Emulsifier HLB value | 0.6 | 0.6 | 0.8 | 0.6 | 0.6 | 0.8 |
| | 1 to 6 | | | | | | |
| | Emulsifier HLB value | 4.0 | 4.0 | 4.0 | 4.0 | 4.0 | 4.0 |
| | 10 to 17 | | | | | | |
| | Crodamol IPIS ¹⁾ | 0.8 | 0.8 | 1.1 | 0.8 | 0.8 | — |
| | Paraffinium Liquidum | — | — | 0.5 | — | — | 5.0 |
| B | Water | to 100 | to 100 | to 100 | to 100 | to 100 | to 100 |
| | Propylene glycol | 2.0 | 2.0 | 2.0 | 2.0 | 2.0 | 2.0 |
| | Sweat-inhibiting active | 26.8 | 26.8 | — | 26.8 | — | — |
| | substance | | | | | | |
| | Deodorizing active | — | — | 2.0 | — | 1.5 | 0.5 |
| | substance | | | | | | |
| C | Non-ionic polyurethane | 1.5 | 2.5 | — | — | 0.5 | 1.5 |
| | polymer A | | | | | | |
| | Non-ionic polyurethane | — | — | 1.5 | 3.8 | 1.5 | — |
| | polymer B | | | | | | |

¹⁾ Emollient; INCI: Isopropyl Isostearate (Croda)

The components of phase A are mixed and heated to 75° C. The aqueous phase B is added slowly with vigorous stirring to the mixture of phase A. Phase C is then added and cooled with stirring to room temperature. The cosmetic agents produced in this way in the form of transparent O/W emulsions have, in spite of the low emulsifier content, a high storage stability and excellent skin compatibility. In addition, the use of these agents leads to a high sweat-inhibiting and/or deodorizing performance and also to a low residue formation on textiles. In addition, these agents have good applicability and dosing accuracy.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent, comprising:
in a cosmetically acceptable carrier and in relation to its total weight,
a) at least one non-ionic polyurethane polymer, comprising at least one polyurethane compound of formula (I) and at least one non-ionic polyether compound of formula (II) and at least one ether compound of formula (III) 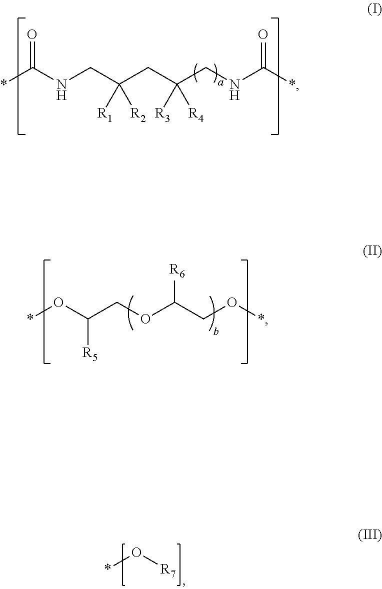
in which
R₁ to R₆, each independently of one another, stand for hydrogen or a linear or branched C₁-C₄ alkyl group,
R₇ stands for a linear or branched C₁₀-C₂₈ alkyl group,
a stands for integers from 1 to 5, and
b stands for integers from 50 to 200,
b) at least one emulsifier having an HLB value from 1 to 6,
c) at least one emulsifier having an HLB value from 10 to 17, and
d) at least one sweat-inhibiting and/or deodorizing active substance,
wherein the total amount of emulsifiers in the cosmetic agent is from 0.01 to 4.9 wt. %.

2. The cosmetic agent according to claim 1, wherein the at least one non-ionic polyurethane polymer comprises, as polyurethane compound of formula (I), at least one polyurethane compound of formula (Ia) or of formula (Ib) and/or of formula (Ic) 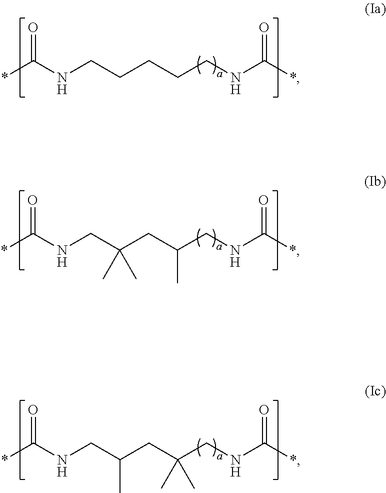
in which
a in each case stands for the integer 2.

3. The cosmetic agent according to claim 1, wherein the at least one non-ionic polyurethane polymer includes, as non-ionic polyether compound of formula (II), at least one non-ionic polyether compound of formula (IIa) 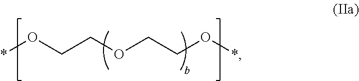
in which
b stands for integers of 60 to 150.

4. The cosmetic agent according to claim 1, wherein the at least one non-ionic polyurethane polymer includes, as ether compound of formula (III), at least one ether compound of formula (IIIa) or formula (IIIb) 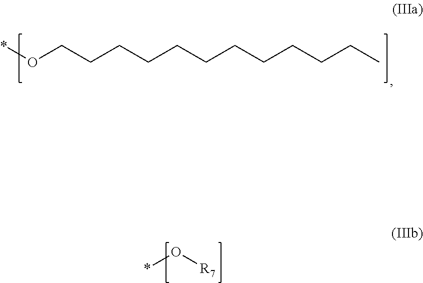
in which
R₇ stands for a branched C16-C20 alkyl group.

5. The cosmetic agent according to claim 1, wherein the at least one non-ionic polyurethane polymer further comprises at least one polyamine compound of formula (IV) (IV) 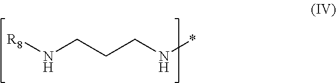
in which
R₈ stands for a linear or branched C8-C20 alkyl group.

6. A cosmetic agent according to claim 1, wherein the at least one non-ionic polyurethane polymer includes at least one polyurethane compound of formula (Ia), at least one non-ionic polyether compound of formula (IIa), at least one ether compound of formula (IIIa), and at least one polyamine compound of formula (IV), 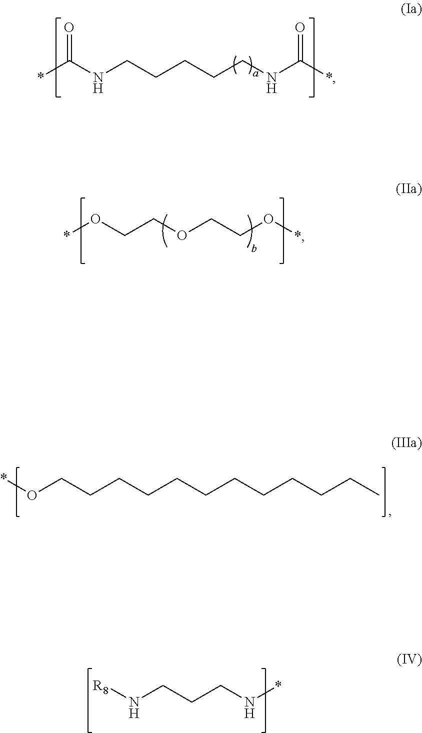
in which
a stands for the integer 2,
b stands for integers of 80 to 110, and
R₈ stands for a linear or branched C8-C20 alkyl group.

7. The cosmetic agent according to claim 1, wherein the at least one non-ionic polyurethane polymer is a copolymer modified terminally with lauryl alcohol and consisting of cocoaminopropylamine, polyethylene glycol having an average of 100 ethylene oxide units and hexamethylene diisocyanate (HDI).

8. The cosmetic agent according to claim 1, wherein the at least one non-ionic polyurethane polymer comprises at least one polyurethane compound of formula (Ib) and/or of formula (Ic), at least one non-ionic polyether compound of formula (IIa) and at least one ether compound of formula (IIIb) 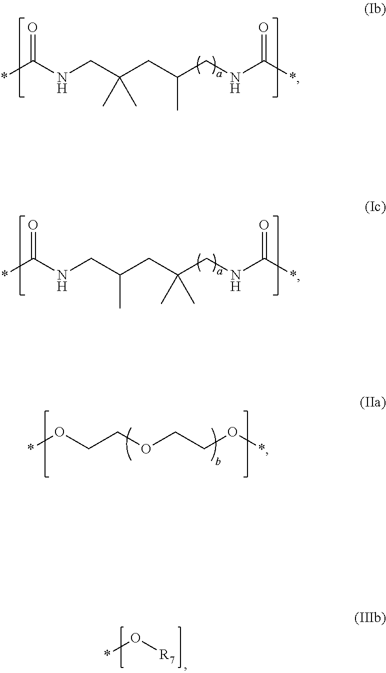
in which
a in each case stands for the integer 2,
b stands for integers of 80 to 110, and
R₇ stands for a branched C16-C20 alkyl group.

9. The cosmetic agent according to claim 1, wherein the at least one non-ionic polyurethane polymer is a copolymer modified terminally with a branched C16-C20 alcohol and consisting of trimethylhexane diisocyanate (TMHDI) and polyethylene glycol having an average of 90 ethylene oxide units.

10. The cosmetic agent according to claim 1, wherein the at least one non-ionic polyurethane polymer is included in a total amount of 0.2 to 5 wt. % based on the total weight of the cosmetic agent.

11. The cosmetic agent according to claim 1, wherein the at least one non-ionic polyurethane polymer is included in a total amount of 1.0 to 4.0 wt. % based on the total weight of the cosmetic agent.

12. The cosmetic agent according to claim 1, wherein the at least one emulsifier having an HLB value of 1 to 6 is included in a total amount of 0.1 to 1.7 wt. % based on the total weight of the cosmetic agent.

13. The cosmetic agent according to claim 1, wherein the at least one emulsifier having an HLB value of 1 to 6 is included in a total amount of 0.4 to 1.0 wt. % based on the total weight of the cosmetic agent.

14. The cosmetic agent according to claim 1, wherein the at least one emulsifier having an HLB value of 10 to 17 is included in a total amount of 1.0 to 4.3 wt. %, based on the total weight of the cosmetic agent.

15. The cosmetic agent according to claim 1, wherein the at least one emulsifier having an HLB value of 10 to 17 is included in a total amount of 2.0 to 4.0 wt. % based on the total weight of the cosmetic agent.

16. The cosmetic agent according to claim 1, wherein the cosmetic agent has a ratio by weight of the at least one emulsifier having an HLB value of 10 to 17 to the at least one emulsifier having an HLB value of up to 6 of 10:1 to 1:5.

17. The cosmetic agent according to claim 1, wherein the cosmetic agent has a ratio by weight of the at least one emulsifier having an HLB value of 10 to 17 to the at least one emulsifier having an HLB value of up to 6 of 7:1 to 4:1.

18. The cosmetic agent according to claim 1, wherein the total amount of emulsifiers in the cosmetic agent is 0.05 to 4.9 wt. % based on the total weight of the cosmetic agent.

19. The cosmetic agent according to claim 1, wherein the cosmetic agent is present in the form of a transparent O/W emulsion having a mean particle diameter D50 of 10 to 100 nm.

20. A non-therapeutic cosmetic method for reducing the perspiration of the body and/or for reducing the body odor triggered by perspiration, comprising: applying to human skin a cosmetic agent according to claim 1 and leaving the cosmetic agent on the human skin for at least 1 hour.
